SEQUENCE LISTING (12 sequences)

SEQ ID NO: 1 - protein, 5 aa, synthetic construct
NYALN

SEQ ID NO: 2 - protein, 17 aa, synthetic construct
WINTYSGVGTYAGEFKG

SEQ ID NO: 3 - protein, 11 aa, synthetic construct
RGFYGRRFFDV

SEQ ID NO: 4 - protein, 15 aa, synthetic construct
KASQSVDYDVDSYMH

SEQ ID NO: 5 - protein, 7 aa, synthetic construct
AASNLAS

SEQ ID NO: 6 - protein, 9 aa, synthetic construct
QQTIEDPPT

SEQ ID NO: 7 - protein, 120 aa, synthetic construct
QVQLVQSGSELKKPGASVKVSCKASGYTFTNYALNWVRQAPGQGLEWMGWINTYSGVGTYAGEFKGRFVFSLDTSVSTAYLQISSLKAEDTAVYYCARRGFYGRRFFDVWGKGTTVTVSS

SEQ ID NO: 8 - protein, 111 aa, synthetic construct
DIQLTQSPSSLSASVGDRVTITCKASQSVDYDVDSYMHWYQQKPGKAPKLLIYAASNLASGVPSRFSGSGSGTDFTFTISSLQPEDIATYYCQQTIEDPPTFGGGTKVEIK

SEQ ID NO: 9 - protein, 447 aa, synthetic construct
QVQLVQSGSELKKPGASVKVSCKASGYTFTNYALNWVRQAPGQGLEWMGWINTYSGVGTYAGEFKGRFVFSLDTSVSTAYLQISSLKAEDTAVYYCARRGFYGRRFFDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 218 aa, synthetic construct
DIQLTQSPSSLSASVGDRVTITCKASQSVDYDVDSYMHWYQQKPGKAPKLLIYAASNLASGVPSRFSGSGSGTDFTFTISSLQPEDIATYYCQQTIEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, synthetic construct [sequence omitted]

(19) United States
(12) Patent Application Publication
(10) Pub. No.: US 2025/0346683 A1
(43) Pub. Date: Nov. 13, 2025

(54) METHODS OF TREATING CORONARY ARTERY DISEASE

(71) Applicant: MedImmune, LLC, Gaithersburg, MD (US)

(72) Inventors: Joseph Samuel Grimsby, Gaithersburg, MD (US); Chao-Yu Jin, Gaithersburg, MD (US); Judith Falloon, Gaithersburg, MD (US); Judith Ann Hsia, Gaithersburg, MD (US); Sotirios K. Karathanasis, Gaithersburg, MD (US); Richard Thomas George, Gaithersburg, MD (US)

(21) Appl. No.: 19/075,308

(22) Filed: Mar. 10, 2025

Related U.S. Application Data
(60) Provisional application No. 63/564,281, filed on Mar. 12, 2024.

(51) Int. Cl. C07K 16/40; A61K 39/00; A61P 9/10

(52) U.S. Cl. CPC C07K 16/40; A61P 9/10; A61K 2039/505; A61K 2039/54; A61K 2039/545; C07K 2317/76

(57) ABSTRACT

This invention relates to methods of lowering plasma levels of endothelial lipase (EL) in patients in need thereof comprising administering MEDI5884 to subjects at a dose of from about 50 mg to about 600 mg per month.

### RELATED APPLICATIONS

This application claims the benefit of and priority to U.S. Provisional Application No. 63/564,281, filed on Mar. 12, 2024, which is hereby incorporated by reference in its entirety.

### SEQUENCE LISTING

This application incorporates by reference a sequence listing submitted as a text file entitled “REGIO-008-01US-ST26.xml” created on Jul. 17, 2025 and having a size of 16 kilobytes.

### FIELD OF THE INVENTION

This invention relates to methods of lowering plasma levels of endothelial lipase (EL) in patients in need thereof comprising administering MEDI5884 to subjects at a dose of from about 50 mg to about 600 mg per month.

### BACKGROUND OF THE INVENTION

Endothelial lipase (EL) is a circulating phospholipase that has been identified as a member of the triglyceride lipase family. EL has both phospholipase and triglyceride lipase activities, and it hydrolyzes high density lipoproteins (HDL) more efficiently than other lipoproteins. It is believed to play a key role in regulating plasma HDL cholesterol (HDL-C) levels. By hydrolyzing HDL-phospholipids, EL causes HDL particle destabilization and rapid clearance by the kidneys.

Increased plasma EL concentrations have been associated with a deteriorated lipoprotein-lipid profile along with elevated plasma triglyceride and apolipoprotein B concentrations, as well as with smaller low density lipoprotein particle size. (Paradis et al., Can J Cardiol 22: 31B-34B (2006)). Elevated proinflammatory cytokine concentrations and an increased prevalence of the metabolic syndrome have also been observed among individuals with elevated plasma EL concentrations. Given these and other factors, EL has been considered to play an important role in cardiovascular disease.

### SUMMARY OF THE INVENTION

This invention relates to methods of lowering plasma levels of endothelial lipase (EL) in patients in need thereof comprising administering MEDI5884 to subjects at a dose of from about 50 mg to about 600 mg per month.

### BRIEF DESCRIPTION OF THE DRAWINGS

FIG. 1 depicts the dose-dependent suppression of plasma endothelial lipase (EL) levels in healthy volunteers (HV) (as a treated population) upon administration of a single subcutaneous dose of MEDI5884. Avg=average; error bars represent SEM=standard error of the mean.

FIG. 2 depicts the dose-dependent suppression of plasma endothelial lipase (EL) levels (change from baseline) in coronary artery disease (CAD) patients upon administration of 3 monthly subcutaneous doses of MEDI5884. Avg=average; error bars represent SEM=standard error of the mean.

FIG. 3 depicts Dose-dependent suppression of plasma endothelial lipase (EL) levels (ng/ml) in coronary artery disease (CAD) patients upon administration of 3 monthly subcutaneous doses of MEDI5884. Avg=average; error bars represent SEM=standard error of the mean.

### DETAILED DESCRIPTION OF THE INVENTION

This invention relates to methods of lowering plasma levels of endothelial lipase (EL) in patients in need thereof comprising administering MEDI5884 to subjects at a dose of from about 50 mg to about 600 mg per month.

As used herein, the term MEDI5884 is an antibody or antigen-binding fragment thereof that is defined by the three heavy chain CDRs provided in Table 1 and three light chain CDRs provided in Table 2.

TABLE 1
VH CDR Amino Acid Sequences*

| | VH CDR1 | VH CDR2 | VH CDR3 |
|---|---|---|---|
| | (SEQ ID | (SEQ ID | (SEQ ID |
| | NO:) | NO:) | NO:) |
| MEDI5884 | NYALN | WINTYSGVG | RGFYGRR |
| | (SEQ ID | TYAGEFKG | FFDV |
| | NO: 1) | (SEQ ID | (SEQ ID |
| | | NO: 2) | NO: 3) |

*The VH CDRs in Table 1 are determined according to Kabat.

TABLE 2
VL CDR Amino Acid Sequences*

| | VL CDR1 | VL CDR2 | VL CDR3 |
|---|---|---|---|
| | (SEQ ID | (SEQ ID | (SEQ ID |
| | NO:) | NO:) | NO:) |
| MEDI5884 | KASQSVDY | AASNLAS | QQTIEDP |
| | DVDSYMH | (SEQ ID | PT |
| | (SEQ ID | NO: 5) | (SEQ ID |
| | NO: 4) | | NO: 6) |

*The VL CDRs in Table 1 are determined according to Kabat.

In some embodiments, the MEDI5884 antibody used in the methods of the present invention is an antibody or antigen-binding fragment defined by the heavy and light variable regions as disclosed in Tables 3 and 4, respectively.

TABLE 3
VH Chain Amino Acid Sequence
MEDI5884 VH Chain

QVQLVQSGSELKKPGASVKVSCKASGYTFTNYALNWVRQAPGQGLEWMGW
INTYSGVGTYAGEFKGRFVFSLDTSVSTAYLQISSLKAEDTAVYYCARRG
FYGRRFFDVWGKGTTVTVSS (SEQ ID NO: 7)

TABLE 4
VL Chain Amino Acid Sequence
MEDI5884 VL Chain

DIQLTQSPSSLSASVGDRVTITCKASQSVDYDVDSYMHWYQQKPGKAPKL
LIYAASNLASGVPSRFSGSGSGTDFTFTISSLQPEDIATYYCQQTIEDPP
TFGGGTKVEIK (SEQ ID NO: 8)

In other embodiments of the present invention, the MEDI5884 is an antibody or antigen-binding fragment thereof comprises the full-length heavy chain amino acid sequence as provided in Table 5 and the full-length light chain amino acid sequence as provided in Table 6.

TABLE 5
Full length Heavy Chain Amino Acid Sequence
MEDI5884 Heavy Chain

QVQLVQSGSELKKPGASVKVSCKASGYTFTNYALNWVRQAPGQGLEWMGW
INTYSGVGTYAGEFKGRFVFSLDTSVSTAYLQISSLKAEDTAVYYCARRG
FYGRRFFDVWGKGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
(SEQ ID NO: 9)

TABLE 6
Full length Light Chain Amino Acid Sequence
MEDI5884 Light Chain

DIQLTQSPSSLSASVGDRVTITCKASQSVDYDVDSYMHWYQQKPGKAPKL
LIYAASNLASGVPSRFSGSGSGTDFTFTISSLQPEDIATYYCQQTIEDPP
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC (SEQ ID NO: 10)

In some aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop. The Kabat numbering scheme places the insertions at H35A and H35B but if neither 35A nor 35B are present, the loop ends at 32. If only 35A is present, the loop ends at 33 and if both 35A and 35B are present, the loop ends at 34.

In some aspects, provided herein are methods of administering antibodies and antigen-binding fragments thereof comprise the Chothia VH and VL CDRs of the MEDI5884 antibody listed in Tables 1 and 2. In some aspects of the present invention, the invention provides methods of administering antibodies or antigen-binding fragments thereof comprise one or more of the CDRs listed in Tables 1 and/or 2, for which the Chothia and Kabat CDRs have the same amino acid sequence. In some aspects of the present invention, provided herein are methods of administering antibodies and antigen-binding fragments thereof comprise combinations of Kabat CDRs and Chothia CDRs.

In some aspects of the present invention, the CDRs of an antibody or antigen-binding fragment thereof can be determined according to the IMGT numbering system as described in Lefranc M-P, (1999) The Immunologist 7:132-136 and Lefranc M-P et al., (1999) Nucleic Acids Res 27:209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects of the present invention, provided herein are methods of administering antibodies and antigen-binding fragments thereof comprise the IMGT VH and VL CDRs of the MEDI5884 antibody listed in Tables 3 and 4, for example, as described in Lefranc M-P (1999) supra and Lefranc M-P et al., (1999) supra).

In some aspects, the CDRs of an antibody or antigen-binding fragment thereof can be determined according to MacCallum R M et al., (1996) J Mol Biol 262:732-745. See also, e.g., Martin A. “Protein Sequence and Structure Analysis of Antibody Variable Domains,” in Antibody Engineering, Kontermann and Dithel, eds., Chapter 31, pp. 422-439, Springer-Verlag, Berlin (2001). In some aspects of the present invention, provided herein are methods of administering antibodies or antigen-binding fragments thereof and comprise VH and VL CDRs of the MEDI5884 antibody listed in Tables 3 and 4 as determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects of the present invention, provided herein are methods of administering antibodies or antigen-binding fragments thereof and comprise VH and VL CDRs of the MEDI5884 antibody listed in Tables 3 and 4 as determined by the AbM numbering scheme.

TABLE 7
Heavy Chain Constant Region
Amino Acid Sequence
MEDI5884 Heavy Chain Constant Region

ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK (SEQ ID NO: 11)

TABLE 8
Light Chain Constant Region
Amino Acid Sequence
MEDI5884 Light Chain Constant Region

RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC (SEQ ID NO: 12)

The MEDI5884 antibody as defined herein may, but need not, specifically bind to human endothelial lipase (EL). Human EL is encoded by the LIPG gene, and the mature form is a 480-amino acid protein. The human EL protein sequence can be accessed on the UniProt Database via accession number Q9Y5X9, the entire record of which is herein incorporated by reference. The methods of the present invention, however, do not necessarily depend on the ability of MEDI5884 to specifically bind to or interact with human EL.

The EL protein is part of the lipase family of circulating proteins in mammals, e.g., humans, that hydrolyse lipoproteins to permit cellular uptake of fatty acids. Thus, generally speaking, higher circulating levels of EL correlate with coronary artery disease (CAD) and other cardiovascular diseases.

According to the present invention, the administration of MEDI5884 to patients results in the lowering of plasma EL levels. In some embodiments, the patients receiving the MEDI5884 administration are CAD patients. In other embodiments of the present invention the patients receiving the MEDI5884 administration have elevated levels of circulating EL, but otherwise exhibit no other symptoms of CAD.

In one embodiment of the present invention, the phrase elevated levels of circulating EL (or elevated levels of plasma EL) may refer to measured levels of EL taken from the patients blood, wherein the levels are higher than those levels of subjects that are considered to have normal levels. In another embodiment of the present invention, the phrase elevated levels of circulating EL (or elevated levels of plasma EL) may also refer to measured levels of EL taken from the patients blood, wherein the levels are higher than those levels measured in the same subject at a previous time point, i.e., the levels of circulating EL have risen over time in the subject. Thus the term normal levels may refer to an established range of concentration (or total mass) of circulating EL in patients that are considered to not have any symptoms of CAD or any other cardiovascular disease, or the term normal levels may refer to an established baseline of a concentration, or range thereof (or total mass) of circulating EL in the patient prior to exhibiting any signs or symptoms of CAD or any other cardiovascular disease. For the purposes of this disclosure, the phase “levels of EL” is understood to mean circulating levels of EL.

Thus, the methods of the present invention also include administering MEDI5884 to patients that have higher circulating EL levels than subjects that do not exhibit any signs of CAD. The terms patient and subject can be used interchangeably herein.

The methods of the present invention are not necessarily dependent upon establishing baseline levels of EL prior to the administration of MEDI5884. Indeed, if the attending healthcare provider has determined that the patient is exhibiting symptoms of or has been diagnosed with CAD, the attending healthcare provider may administer or cause to be administered the MEDI5884 therapeutic treatment.

In some embodiments of the present invention, the MEDI5884 is administered to the patient once a month. In additional embodiments, the MEDI5884 is administered once a month for at least 2, 3, 4, 5, 6, 7, 8, 9, 10, 11, 12, 13, 14, 15, 16, 17, 18, 19, 20, 21, 22, 23, 24 months or more. In still more embodiments, the MEDI5884 maybe administered once a month as a maintenance therapy to the patient for the life of the patient. In still more embodiments, the MEDI5884 is administered once every other month or once every three months. Similarly, if the MEDI5884 is administered less frequently than once a month, the MEDI5884 may still be administered as a maintenance therapy to the patient on a schedule set by the attending healthcare provider.

In other embodiments, the MEDI5884 is administered subcutaneously to the patient. In still other embodiments, the MEDI5884 is administered intravenously, intraarterially, intramuscularly, intradermally or intraperitoneally to the patient. The methods of administration need not be identical for each administration of MEDI5884 to the patient.

The MEDI5884 may be administered to the patient in a variety of doses per administration. Moreover, each administration of MEDI5884 need not be identical. For example, the attending healthcare provider may increase the dose of MEDI5884 per administration if the levels of circulating EL do not exhibit signs of falling to normal levels. Accordingly, the methods of the present invention further comprise increasing the dose of MEDI5884 in the patient when the levels of plasma EL are not below an established normal level of EL in the patient after the patient has received at least dose of MEDI5884.

The dose of MEDI5884 per administration may be from about 10 mg to about 1000 mg, or any dose in between. The dose of MEDI5884 per administration may be from about 15 mg to about 950 mg, or from about 20 mg to about 900 mg, or from about 25 mg to about 850 mg, or from about 30 mg to about 800 mg, or from about 35 mg to about 750 mg, or from about 40 mg to about 700 mg, or from about 45 mg to about 650 mg, or from about 50 mg to about 600 mg, or from about 50 mg to about 550 mg, or from about 50 mg to about 500 mg, or from about 50 mg to about 450 mg, or from about 50 mg to about 400 mg. In specific embodiments, the dose of MEDI5884 administered may be about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg or about 750 mg.

### EXAMPLES

### Example 1

The healthy volunteers included males and females of nonchildbearing potential between the ages of 18 and 55 years, with a body mass index of between 18 and 30 kg/m2 at the time of screening. The volunteers were randomized into groups of at least 12, placebo-controlled, and blinded to both the participants and investigators.

The coronary artery disease (CAD) patients were between 45 and 80 years of age and had clinically evident coronary artery disease, defined as a history of myocardial infarction, coronary revascularization, coronary atherosclerosis diagnosed >3 months before screening (based on invasive or noninvasive testing), or abnormal stress testing diagnostic of coronary artery disease. Patients had to have an LDL-C≤100 mg/dl on a stable dose (≥28 days before screening) of high-intensity statin therapy defined as atorvastatin ≥40 mg or rosuvastatin ≥20 mg daily for non-Asian patients, or ≥10 mg rosuvastatin daily for Asian patients. Exclusion criteria included triglycerides >500 mg/dl and any unstable cardiovascular condition within the past 3 months.

Sandwich immunoassay was developed for the detection human EL utilizing ECL readout. An MSD (MesoScale Discovery) plate was coated with 5 μg/ml of MEDI5884, at 50 μl/well, incubated at 4° C. on a flat surface and subsequently washed 3× with 300 μl/well of ELISA Wash Buffer. The assay plate(s) were blocked with 150 μl/well of I Block Buffer (IBB) for ≥60 min on an orbital plate shaker at room temperature (RT). Reference standards (RS), quality control (QC) and negative control (NC), prepared in IBB, and test samples were added to the plates at 35 μl/well. Samples that had been previously tested determined the minimally required dilution ratio (MRD) as two (2) for plasma samples. The assay plate(s) were incubated at RT on a plate shaker with gentle shaking for 60 minutes±10 minutes. Unbound analyte was removed by washing the plate(s) with ELISA wash buffer. To detect the captured analyte, 1 μg/ml of biotinylated anti-human EL monoclonal antibody (OriGene) was added at 35 μl/well and incubated for an additional 60 minutes±10 minutes at RT on a plate shaker. Unbound detection antibody was removed by washing the plate(s). Streptavidin Sulfo-TAG was added at 35 μl/well and incubated for additional 60 minutes±10 minutes at RT on a plate shaker with gentle shaking. Plates were washed again and Read Buffer was added at 150 μl/well. Signal was read on a MSD sector imager instrument within 20 minutes of addition of the Read Buffer. The ECL values for each plate were collected using the MSD Sector Imager. The ECL values for the reference standard curves for each plate were plotted with Softmax Pro GxP v6.4 software (Molecular Devices, Sunnyvale, CA) using a 1/γ2-weighted 4-Parameter Logistic (4-PL) model of curve fitting. The concentrations of unknown samples were interpolated from the respective standard curves.

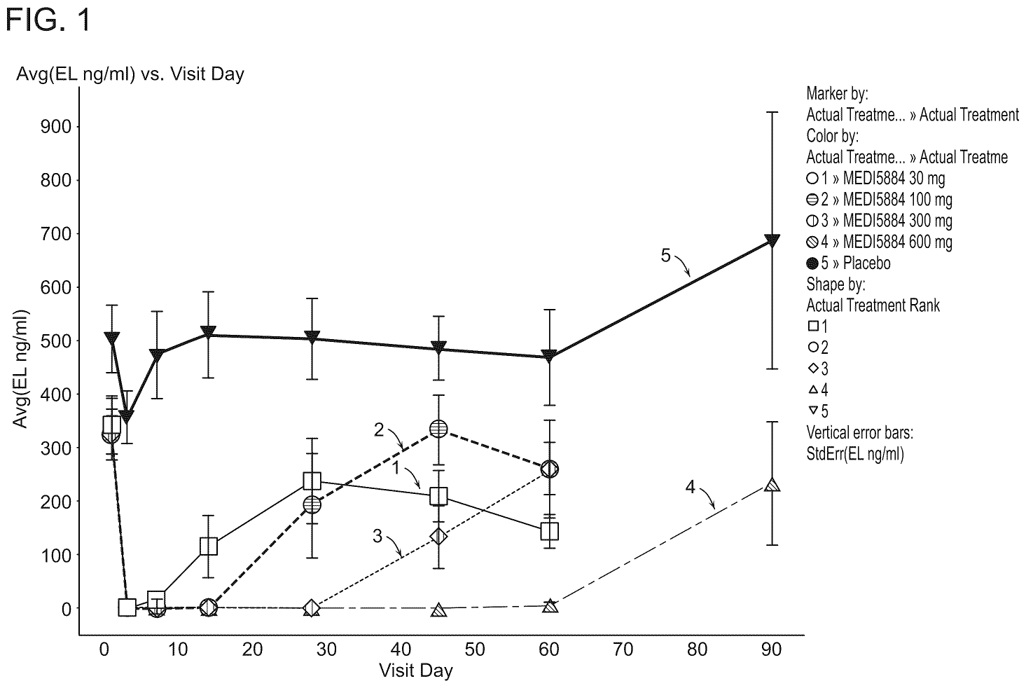

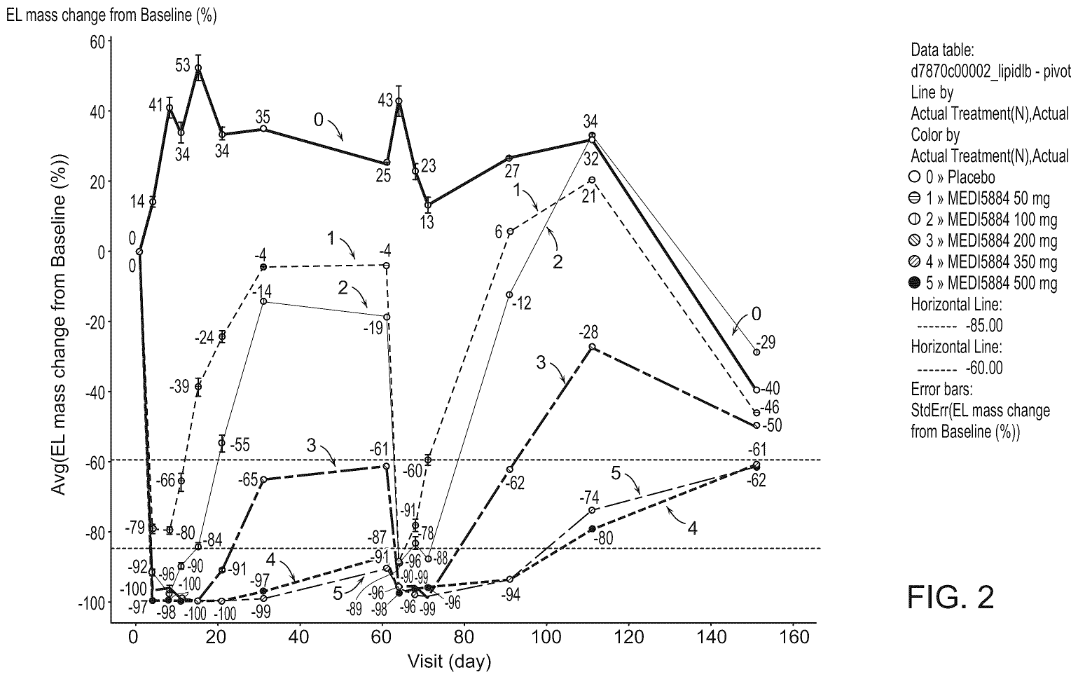

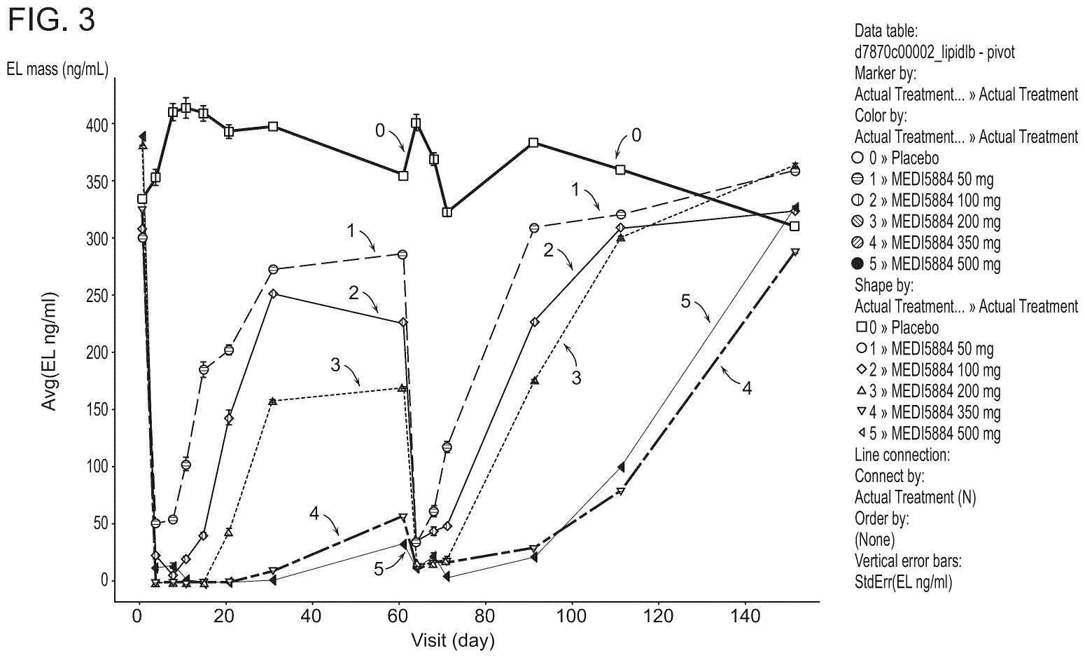

Following administration of MEDI5884, dose-dependent suppression of EL levels was observed (FIGS. 1, 2 and 3). Complete suppression of EL levels was observed for up to 45 days post dosing for the MEDI5884 300 and 600 mg dose groups for HV. In CAD subjects, after 3 monthly subcutaneous MEDI5884 doses, dose-dependent suppression of EL mass was observed. EL mass decreased >85% from baseline through Day 91 at MEDI5884 350 and 500 mg doses. EL mass decreased >60% from baseline through Day 91 at MEDI5884 200 mg. Initially, 100 mg dose suppressed EL almost completely, while 50 mg dose achieved approximately 80% suppression. Subsequently, EL suppression reversed consistently as MEDI5884 exposure decreased, in a dose-dependent manner.

What is claimed is:

1. A method of lowering plasma levels of endothelial lipase (EL) in a patient in need thereof comprising administering MEDI5884 to the subject at a dose of from about 50 mg to about 600 mg per month.

2. The method of claim 1, wherein the dose of MEDI5884 is administered once a month.

3. The method of claim 2, wherein the dose of MEDI5884 is administered subcutaneously to the patient.

4. The method of claim 3, wherein the patient has higher EL levels than subjects that do not exhibit any signs of coronary artery disease (CAD).

5. The method of claim 1, further comprising increasing the dose of MEDI5884 in the patient when the levels of plasma EL are not below XXX in the patient after the patient has received at least dose of MEDI5884.
